(19) United States
(12) Patent Application Publication
(10) Pub. No.: US 2018/0343847 A1
(43) Pub. Date: Dec. 6, 2018

(54) DEVICES AND METHODS FOR DISINFECTION AND EXTERMINATION USING UVC LIGHT

(71) Applicant: Keith Ervin, Lakeland, FL (US)

(72) Inventor: Keith Ervin, Lakeland, FL (US)

(21) Appl. No.: 15/992,952

(22) Filed: May 30, 2018

Related U.S. Application Data
(60) Provisional application No. 62/512,970, filed on May 31, 2017.

(51) Int. Cl. A01M 1/22; A01M 21/04; A61L 2/10; B64C 39/02

(52) U.S. Cl. CPC A01M 1/226; A01M 21/046; B64C 2201/12; B64C 39/024; A01M 2200/012; A61L 2/10

(57) ABSTRACT

A method for exterminating a target on a host using ultraviolet (UVC) light. The method includes providing a device configured for emitting UVC light for exterminating the target, selecting a host type from a plurality of host types for the target to be exterminated from, and selecting a target type from a plurality of target types for the target to be exterminated. The method further includes assigning an intensity and an exposure time of UVC light to exterminate the target, wherein the intensity and the exposure time are based at least in part on the host type selected and the target type selected, and wherein the intensity and exposure time together define an extermination program. The method further includes controlling the device to emit UVC light to exterminate the target according to the extermination program.

### CROSS REFERENCE TO RELATED APPLICATIONS

This application claims the benefit of U.S. Provisional Patent Application No. 62/512,970, filed May 31, 2017, which is incorporated herein by reference in its entirety.

### FIELD

The present disclosure generally relates to devices and methods for exterminating targets using UVC light, and more particularly to devices and methods for providing optimized exposure for exterminating targets using UVC light.

### BACKGROUND

The Background and Summary are provided to introduce a foundation and selection of concepts that are further described below in the Detailed Description. The Background and Summary are not intended to identify key or essential features of the potentially claimed subject matter, nor are they intended to be used as an aid in limiting the scope of the potentially claimed subject matter.

UV light, and a particularly UV wavelength C (UVC) light, for example with wavelengths between 240 nanometers-280 nanometers, is known for antimicrobial properties and is used in some settings for disinfection and/or sterilization. However, the present inventor has identified shortcomings with devices and methods presently known in the art, which are addressed through the devices and methods disclosed herein below.

### SUMMARY

Certain embodiments of the present disclosure generally relate to a method for exterminating a target on a host using ultraviolet (UVC) light. The method includes providing a device configured for emitting UVC light for exterminating the target, selecting a host type from a plurality of host types for the target to be exterminated from, and selecting a target type from a plurality of target types for the target to be exterminated. The method further includes assigning an intensity and an exposure time of UVC light to exterminate the target, wherein the intensity and the exposure time are based at least in part on the host type selected and the target type selected, and wherein the intensity and exposure time together define an extermination program. The method further includes controlling the device to emit UVC light to exterminate the target according to the extermination program.

Another embodiment generally relates to device for exterminating a target on a host using ultraviolet (UVC) light. The device includes a UVC emitter configured for emitting UVC light for exterminating the target. A memory module stores a plurality of host types for the target to be exterminated from, a plurality of target types for the target to be exterminated, and a plurality of intensities and exposure times for the UVC emitter to emit UVC light. A user input module is configured for selecting a host type from the plurality of host types and a target type from the plurality of target types. A processing module assigns an intensity and an exposure time of UVC light from the plurality of intensities and exposure times, respectively, based at least in part on the host type selected and the target type selected. The intensity and the exposure time of UVC light assigned together define an extermination program for exterminating the target. A controller module controls the device to emit UVC light to exterminate the target according to the extermination program. An indicator indicates when the device has completed the extermination program.

### BRIEF DESCRIPTION OF THE DRAWINGS

FIG. 1 is a side view of an exemplary embodiment of a hand-held UVC disinfection device.

FIGS. 2A and 2B depict alternative exemplary top views of the device of FIG. 1.

FIG. 3 is an exemplary bottom view of the device of FIG. 1.

FIGS. 4-9 depict a further exemplary embodiment of a hand-held UVC disinfection device.

FIG. 10 is an exemplary side perspective view of an additional exemplary embodiment of a hand-held UVC disinfection device.

FIG. 11 is a front view of the device of FIG. 10.

FIG. 12 is a back view of the embodiment of the device of FIG. 10.

FIG. 13 is a top view of an exemplary embodiment of an aerial drone for UVC disinfection.

FIG. 14 is a side view of the device of FIG. 13.

FIG. 15 is a detailed bottom view of an exemplary embodiment of a foot of a drone as depicted in FIG. 14.

FIG. 16 is a side view of an exemplary embodiment of a terrestrial drone for UVC disinfection.

FIG. 17 is an exemplary bottom view of the drone of FIG. 16.

FIGS. 18-20 depict a still further exemplary embodiment of an aerial drone in the form of an electro-mechanical insect.

FIG. 21 depicts an exemplary method for UVC disinfection using a UVC disinfection device.

FIG. 22 depicts an exemplary program for delivering UVC disinfection.

FIG. 23 depicts an exemplary control system for delivering the UVC disinfection, including through execution of the program of FIG. 22.

### DETAILED DISCLOSURE

The management of pests and diseases is essential to the production of plants, such as crops, as well as animals, and other susceptible goods. The present inventor has identified that in many cases, this management amounts to the use of harmful chemicals. These chemical treatments may be harmful to the underlying good, also referred to as a host, and further harmful to the eventual consumer of that good, not to mention the soil or other assets within the treatment environment. Through experimentation and development, the present inventor has identified that the use of UVC technology may effectively provide protection for these goods without the need to resort to harmful chemical treatments. For example, UVC light may be used to protect plants from harmful microorganisms and disease, including, but not limited to mold, powdery mildew, thrips, white flies, and fungus. When provided in the appropriate dose, or in other words the appropriate intensity and exposure time, UVC wavelength light can effectively kill these and other microorganisms, but notably without causing harm to the plant or other host.

However, the present inventor has also identified that UVC technology presently known in the art does not provide for this appropriate dosage, which depends upon the pest or disease, also referred to as the target, as well as the host on which the target is to be exterminated. In other words, devices presently known in the art do not ensure that a minimum level of UVC light is delivered such that the target is eliminated, nor a maximum level of UV light projected against the host in order to prevent the host from receiving an excessive level that would cause damage thereto. Accordingly, the present inventor has developed the methods and devices of the present disclosure, which may be programmed with procedures or routines of operation for the UV light intensity and/or duration in order to achieve the desired exposure for extermination, without causing an undue or excessive harm to the underlying host.

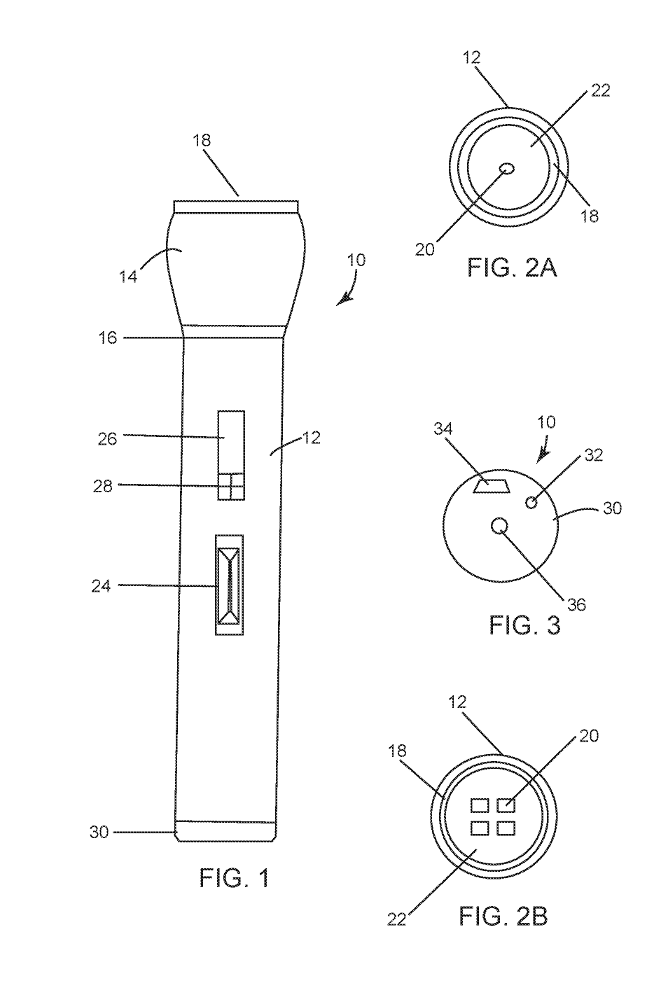

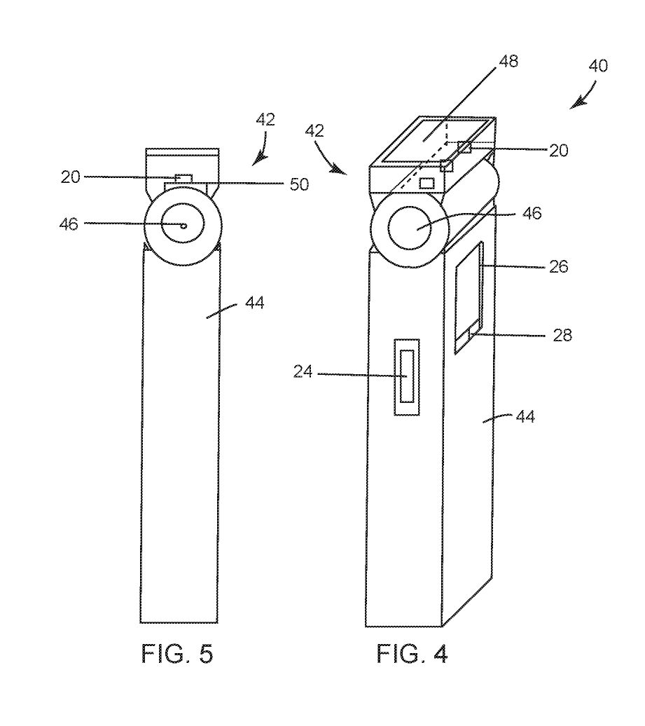

FIG. 1 depicts an exemplary embodiment of a hand-held disinfection device 10 for exterminating a target on a host using ultraviolet (UV) light. The device 10 includes a body 12, which may exemplarily be in the form of a tube and may be constructed of machined metal such as aluminum, for example. A head 14 is secured to the body 12 at a threaded connection 16. The head 14 includes a parabolic lens 18 that is configured to focus the UVC light emitted from one or more UVC wavelength LEDs contained within. It should be recognized that UV emitters other than LEDs are also anticipated by the present disclosure. FIGS. 2A and 2B depict two exemplary and non-limiting embodiments of LED arrangements. For example, FIG. 2A depicts a single LED 20, while FIG. 2B depicts an array of four LEDs 20. A reflective parabolic surface 22 further surrounds the LEDs 20 to direct the UVC light out of the parabolic lens 18. The threaded connection 16 enables the selective removal of the head 14 in order to access the interior of the head 14 and for example LEDs 20.

Returning back to FIG. 1, a power button 24 provides intuitive and simple actuation of the hand-held device. An LCD display 26 and a user input 28, for example buttons, are provided to convey additional information regarding the operation of the device to the user, as well as to prompt and receive user inputs regarding operational controls. For example, the user may use user input device 28 and the LCD display 26 (or any of a variety of other known digitally operated displays) to select the intensity and/or exposure time, select a protocol or routine for operation of the hand-held disinfection device 10, or program the device to create a new protocol or routine. These protocols, routines, and programs are also referred to herein as extermination programs. An operation protocol or routine for the disinfection device 10 may include, but is not limited to, a sequence of time and/or intensity of UVC light emitted by the disinfection device 10. The protocol may execute upon actuation of the power button 24. In other embodiments, this is activated automatically, such as upon programming or detection of a target to be disinfected or exterminated from a host.

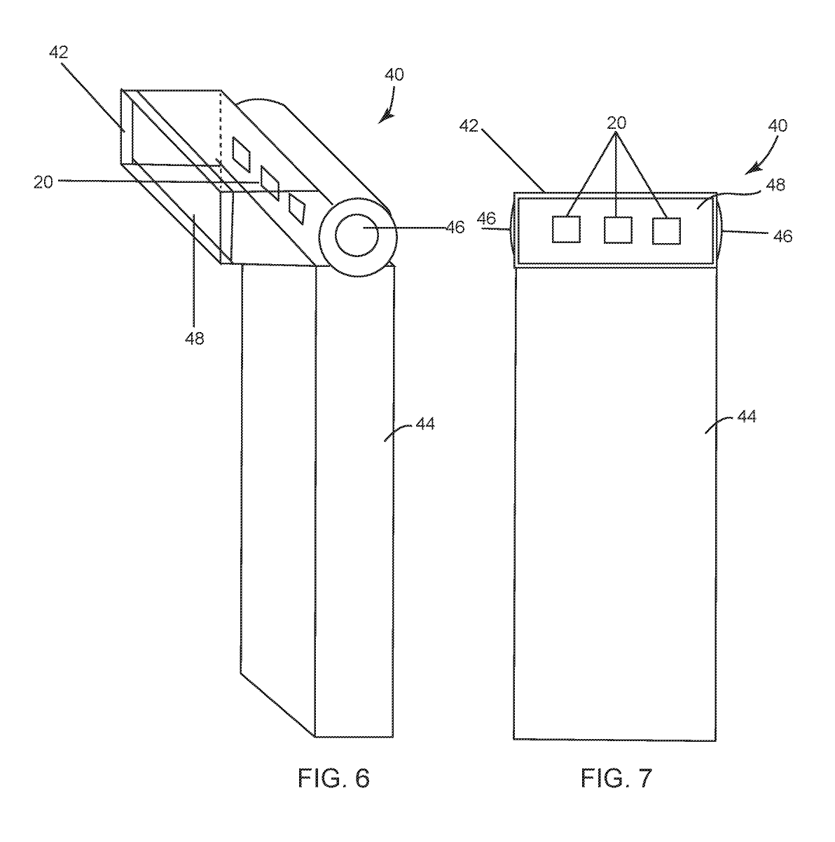

A threaded bottom cap 30 exemplarily selectively closes the interior of the body 12, for example for insertion of replaceable batteries. The bottom cap 30 is further depicted in the bottom view of FIG. 3. In the bottom cap 30, an LED power indicator 32 may be included to illuminate when the device is in operation or has sufficient battery strength for operation. In certain embodiments, a completion indicator (which may also be incorporated with the LED power indicator 32, indicates when the extermination program has been completed for a target. In certain embodiments, one or more electrical connection ports is also provided. For example, a mini or micro USB connection 34 is provided as a device 10 connection for electrical transfer, such as for charging, data transfer for download or upload of data to and from the device 10, or to update the firmware of the device 10, for example with drivers for the display or user input, or to update protocols or routines for operation of the device 10. An electrical port 36 may also be provided, for example in a format suitable to connect to a 3.5 mm electrical jack. In a further embodiment, this port may be used to provide power to the device 10, either for operation, and/or charging an internal battery in the event that a chargeable battery is used within the device.

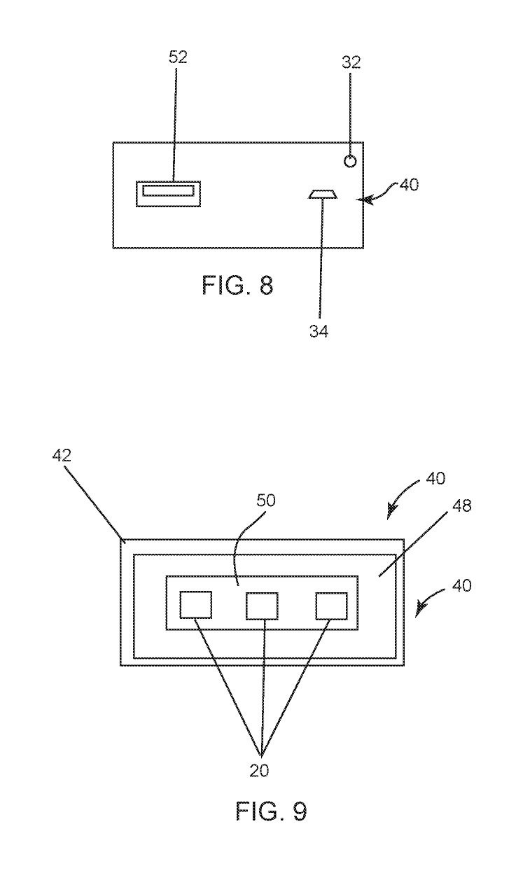

FIGS. 4-9 depict a further exemplary embodiment of a UVC disinfection device 40. It will be recognized that like reference numerals are used herein to reference similar structures between embodiments and that a person of ordinary skill in the art will recognize other combinations of elements described with respect to various embodiments to arrive at further embodiments within the scope of the present disclosure. The device 40 exemplarily includes a moveable head 42. The moveable head is exemplarily secured to the body 44 by a pivotable joint 46. While a rotatable hinge is exemplarily depicted in the described embodiment, other forms of moveable joints, including, but not limited to ball joints or flexible joints, may also be used.

The head 42 includes a lens 48 arranged above an array of LEDs 20. As depicted in FIG. 5, the head 42 further includes printed circuit board assembly 50 that is electronically connected to the LEDs 20 and includes the drivers for the LEDs 20, and that controls the provision of power and illumination of the LEDs 20.

In use, the head 42 is pivotable about the pivotable joint 46 in order to orient the head so as to direct the UVC wavelength light emitted from the LEDs 20 and the head 42 at the intended target. This may further help to limit or avoid exposure of the subject, also referred to herein as the host, and/or to other plants or animals in the vicinity, while ensuring that the UVC wavelength light is properly illuminating the desired treatment areas. In still further embodiments, this may also help to limit exposure of UVC light to the operator by better directing the head 42 away from the operator or a sensitive or unprotected portion of the operator's body, e.g. the operator's eyes.

FIG. 6 is a perspective view of the device 40 with the head 42 rotated. FIG. 7 is a front view of the same configuration of the device 40. FIG. 9 is a top view of the device 40 with the head 42 in the vertical or neutral orientation and FIG. 8 is a bottom view of the device 40 in the same configuration. The device 40 further exemplarily includes full size USB port 52 in addition to a micro USB port 34. The USB and micro USB connections may exemplarily be used for charging a battery internal to the device 40, and/or to provide electronic communicative access to memory internal to the device, which may exemplarily contain status information for example diode life, system usage, or other information. Examples of other information include educational and marketing platforms or audio, video, or text content, reordering or replacement part information, and other content. In a still further exemplary embodiment, wireless communication, for example, according to the Bluetooth protocol, may be used for such communicative connection rather than USB or micro USB.

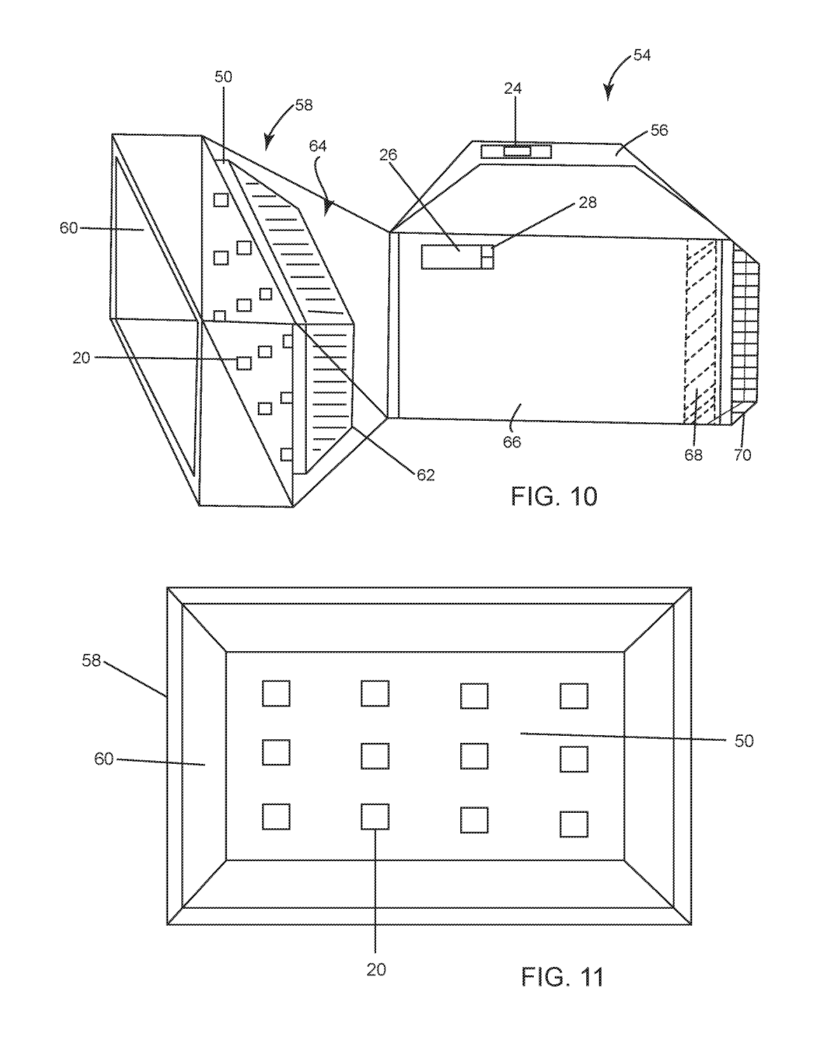

FIGS. 10-12 depict a still further exemplary embodiment of a hand-held UVC disinfection device 54. FIG. 10 is a perspective side view of an exemplary embodiment of the device 54. It exemplarily includes a handle 56 for gripping by a user and the power button 24 is located on the handle 56. The visual display 26 and the user input device 28 operate in the manner as disclosed above to control the operation of the device 54. The device 54 includes a head 58 that contains a plurality of UVC LEDs 20 which are operated by a printed circuit board (PCB) assembly/LED driver 50 to illuminate the LEDs 20, to project UVC wavelength light through a lens 60 at the front of the head 58. A heat sink 62, exemplarily with a plurality of fins, is positioned in proximity to the PCB 50 to draw heat away from the PCB 50 and LEDs 20. A vent 64 may include a space surrounding the heat sink 62 and may include an aperture (not depicted) to the exterior of the device 54. The body 66 of the device 54 exemplarily contains a battery (not depicted) for powering the device 54. In certain embodiments, the battery is a rechargeable battery pack, whereas others accept the insertion of rechargeable or non-rechargeable batteries of common sizes, such as AA, AAA, C, D, or 9-volt, for example. The body 66 further includes a fan 68 that is exemplarily operated by the battery to draw air out of the device through vents 70 so as to cool the heat sink 62 and the rest of the device 54 during operation.

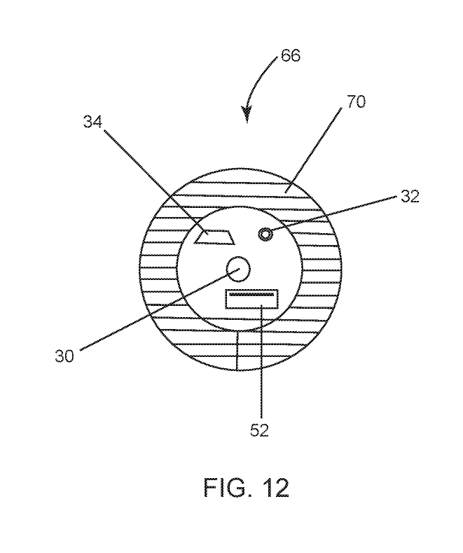

FIG. 11 is a front view of an exemplary embodiment of the device 54. In the front view of FIG. 11, it can be seen that an array of twelve LEDs 20 are depicted. However, it will be recognized by a person of ordinary skill in the art that any number of LEDs 20 may be used in an array while remaining within the scope of the present disclosure.

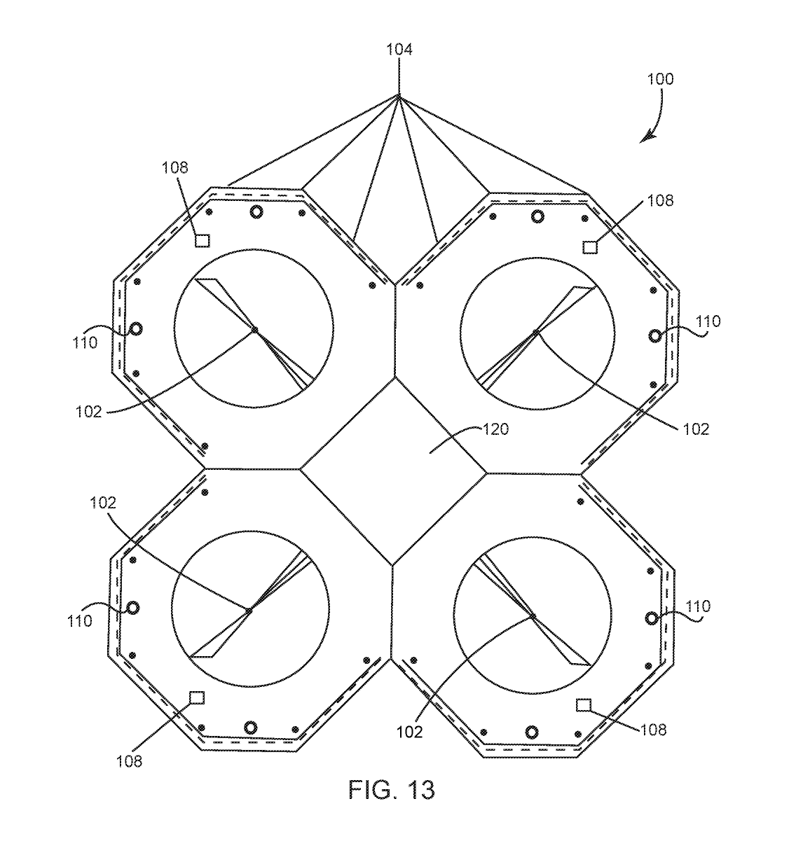

FIG. 12 is a back view of an exemplary embodiment of the device 54, which exemplarily depicts the vents 70, and LED indicator light 32 and various electronic energy and or communication connections. These connections include, but are not limited to USB 54, micro USB 34, and 3.5 mm electrical port 36 connections.

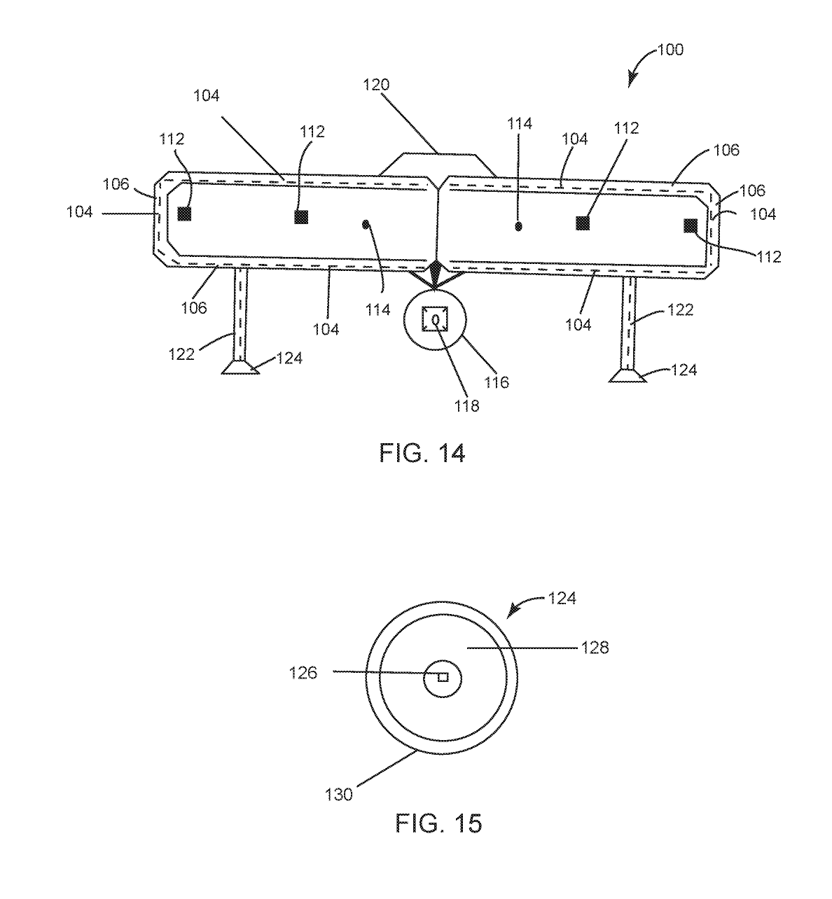

FIG. 13 is a top view of an exemplary embodiment of a drone 100, exemplarily an unmanned aerial vehicle (UAV) drone 100. The drone 100 includes a plurality of props 102 that rotate to propel the drone 100 under user and/or computer operation to move about in the air in the manner known in the art of drones.

The drone 100 includes a plurality of UVC LEDs as will be explained in further detail herein. The UVC LEDs may be arranged in edge arrays 104 to direct UVC light outwardly to the side, up, and/or down. The edge arrays 104 may each include a plurality of LEDs and exemplarily be arranged on a beveled edge 106 surrounding the body of the drone 100. The drone 100 exemplarily includes a plurality of top LEDs 108 that direct UVC light energy above the drone 100. It should be recognized that depending on the potential target and host, along with the desired location of the drone 100 for delivering the UVC light and thus, the extermination program thereto, the top LEDs 108 may also or alternatively be positioned on the bottom of the drone 100 to direct the UVC light energy downwards.

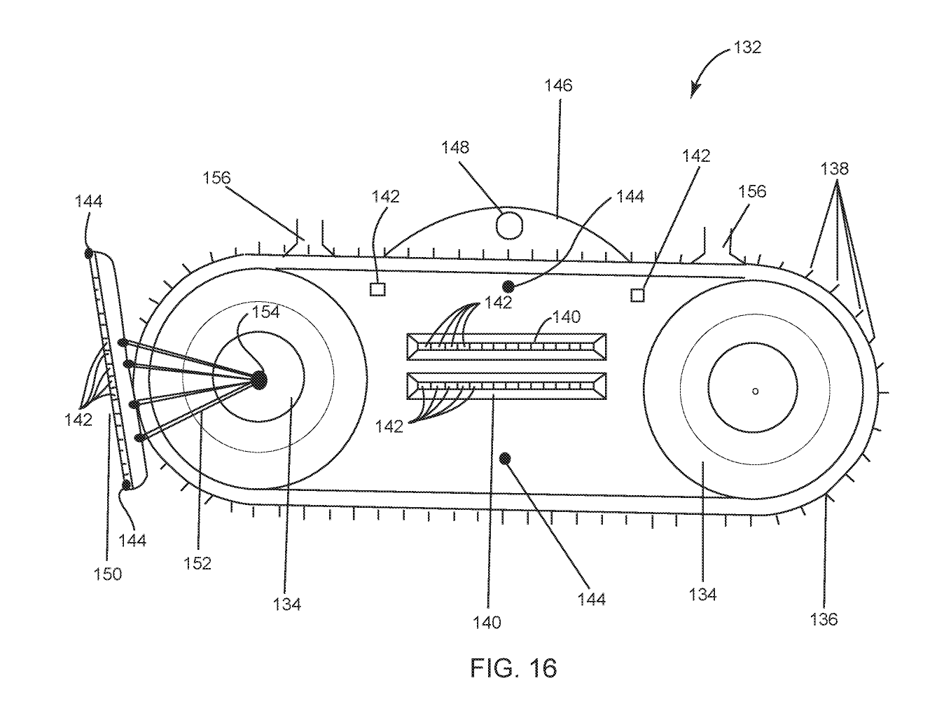

The drone 100 further includes a plurality of video sensors 110 that provide image data regarding the surroundings of the drone 100, which can help for automated navigation or automated navigation assistance for operation of the drone 100. As with the top LEDs 108 previously discussed, the video sensors 110 of other embodiments are alternatively, or additionally, provided on the bottom of the drone 100. FIG. 14 is an exemplary side view of the drone 100. The drone 100 further includes lateral LEDs 112 which project UVC LED laterally from the sides of the drone 100. Video sensor or sensors 110 are directed laterally outward from the drone 100 to additionally aid in automated navigation or navigation assistance. The drone 100 may further include LEDs arranged on the bottom of the drone (not depicted), as discussed above. The bottom of the drone 100 may also include video sensor or sensors as described above, but not depicted in the Figures.

The drone 100 further includes an articulable camera 116. The articulable camera 116 can gather video image data for navigation assistance and/or detection of targets and hosts. Additionally, the articulable camera 116 further may include a UVC LED or LED array 118 that can also be articulated so as to provide further directed UVC exposure to locations identified by the drone 100 or the drone user.

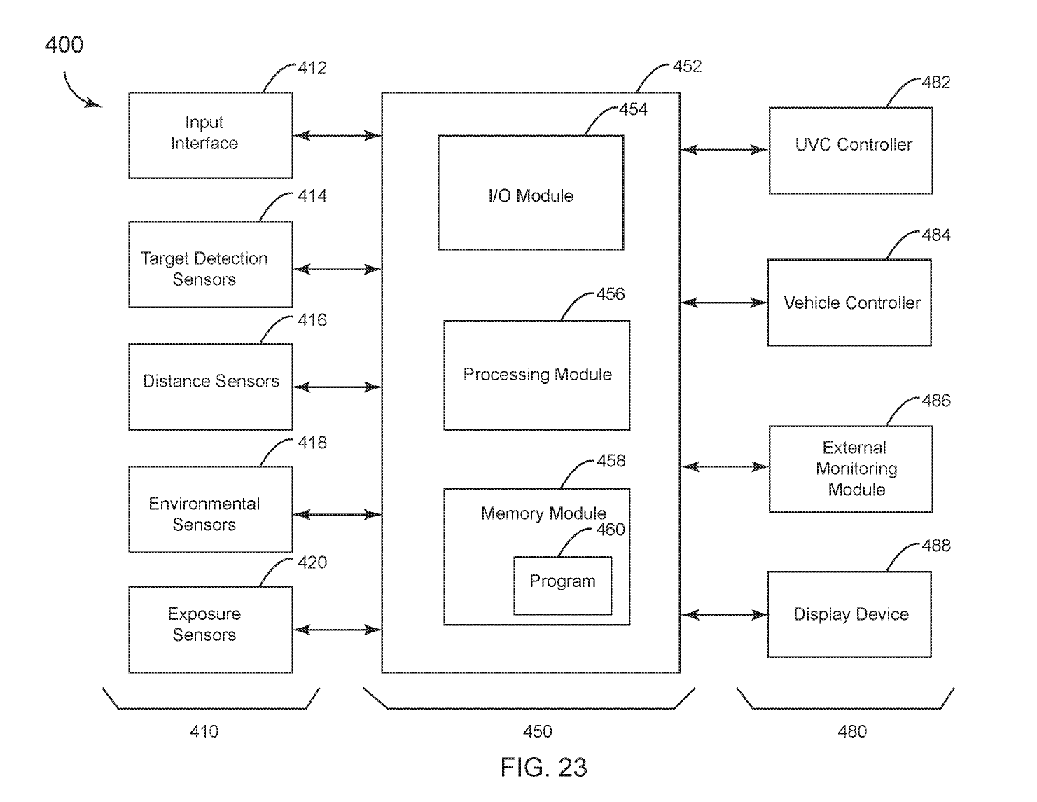

The drone 100 further includes a cockpit 120, which may exemplarily be domed, and house a GPS sensor, controls, microcontrollers, and/or processors for operation of the drone 100, and accelerometers to provide feedback to assist with drone operation and control. These elements of the control system 400 are shown in FIG. 23 and discussed further below.

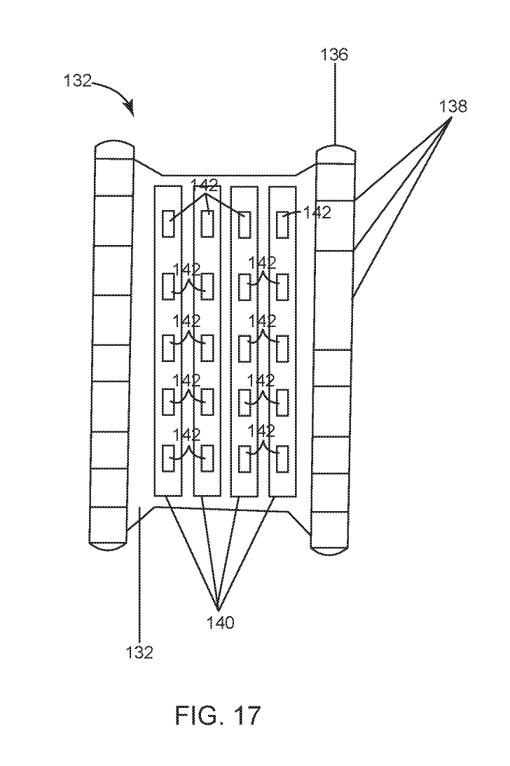

The drone 100 further includes legs 122. FIG. 15 is a detailed bottom view of an exemplary embodiment of a foot 124 at the bottom of an exemplary leg 122. The foot exemplarily includes a UVC LED 126 that is surrounded by a reflective surface 128 and directed outwards through a parabolic lens 130. In such manner, the drone 100 can further use the UVC energy emitted from the feet 124 of the drone 100 for additional directed UVC exposure.

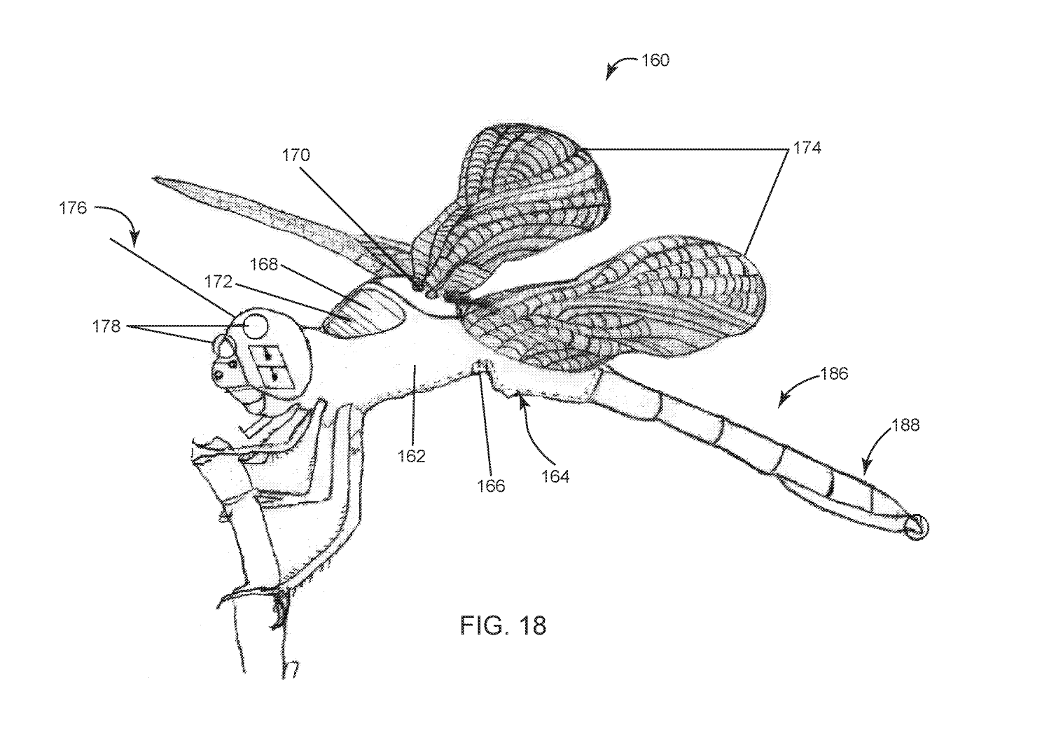

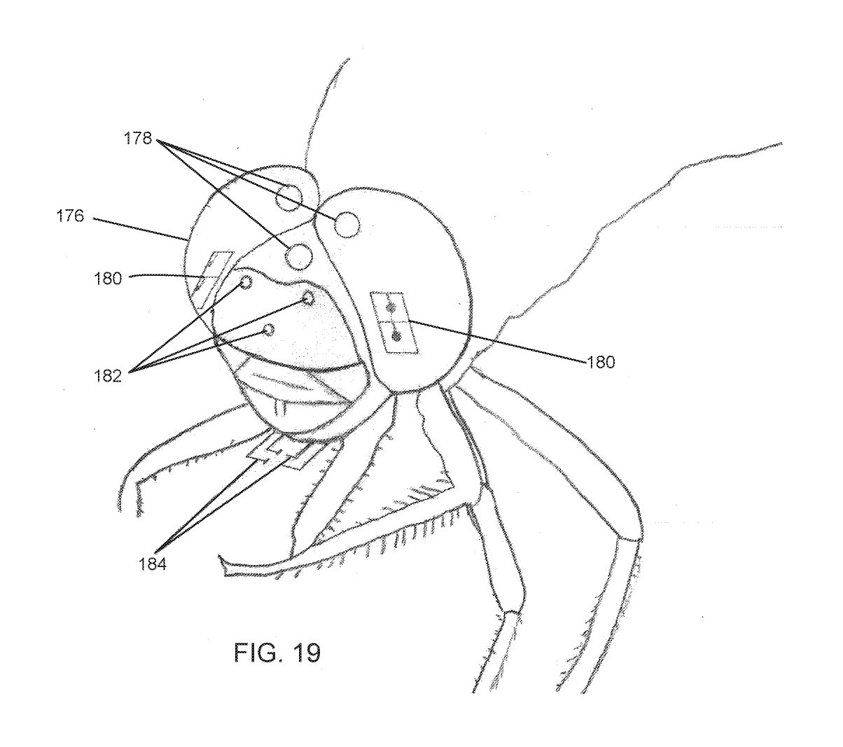

FIGS. 16 and 17 depict an exemplary embodiment of a terrestrial drone 132. The terrestrial drone exemplarily includes wheels 134 that operate to move a track 136 with treads 138 to control movement of the drone 132 over the ground or a surface. The drone 132 includes arrays 140 of LEDs 142 arranged along the sides thereof. The drone 132 includes one or more photo spectroscopic sensors among the sensors 144. These can operate to measure microbial load in an area and may be used to control or select protocol(s) used by the drone 132. It should be recognized that the sensors 142 are also integrated into other embodiments of drones 100, hand-held devices 10, and other devices according to the present disclosure. Video sensors among the sensors 144 provide video or other image data regarding the surrounds of the drone 132 to a controller or processor (not depicted) for use in the operation and control of the drone 132.

The drone 132 further includes a dome 146. An articulable UVC LED emitter 148 is located within the dome 146 and is moveable within the dome 146 to direct UVC light energy at particular directed locations as identified by the operation of the drone 132.

The drone 132 further includes an LED panel 150 that in certain embodiments is movably mounted to the front of the drone 132. The LED panel 150 is exemplarily movably secured to the drone 132 by a plurality of movable panel supports 152 that are connected to the drone 132 at a panel pivot 154. The LED panel 150 and the panel supports 152 are rotatably moveable about the panel pivot 154. In addition to LEDs 142, the LED panel 150 may further include video sensors 144 that operate to collect video image data regarding the surroundings of the drone 132.

The drone 132 further includes UAV landing pads 156, which are exemplarily configured to receive the legs and/or feet of the UAV drone 100, exemplarily depicted in FIGS. 13-15. In an exemplarily embodiment, not only may the UAV drone 100 be releasably secured to the drone 132 for transport to a location for deployment, but in an exemplary embodiment, the UAV landing pads 156 may further provide an electrical connection to charge the UAV drone 100 by any known charging technologies.

FIG. 17 depicts a bottom view of the drone 132 which exemplarily shows an array 140 of UVC LEDs 142 arranged on the bottom of the drone 132 in the area between the tracks 136. In this manner, the drone 132 projects UVC light energy on the ground or surface upon which the drone 132 is driving over.

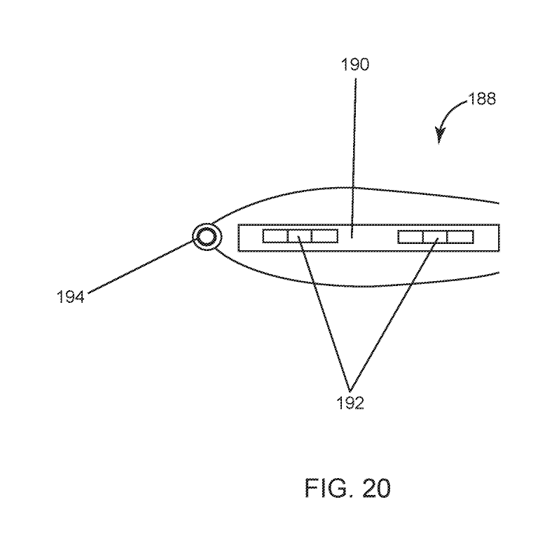

FIGS. 18-20 depict a still further exemplary embodiment of a drone 160. The drone 160 depicted in FIGS. 18-20 is exemplarily embodied in the form factor of an electromechanical insect. The drone 160 includes an abdomen 162 that forms the body of the drone 160 and exemplarily houses the battery and mechanical controls for the drone 160. A plurality of UVC LEDs 164 are arranged about the abdomen 162 and directed outwardly therefrom. In an exemplary embodiment, the UVC LEDs 164 are arranged across the “belly” and the “back” of the abdomen 162. A charging port 166 is exemplarily provided in the abdomen 162, though this may be provided elsewhere on the drone 160.

The mechanical components 168, exemplarily an electrical mechanical motor and gears configured to operate the rotors 170, are further located within the abdomen 162. A plurality of vents 172 are provided in certain embodiments to dissipate any excess heat generated in the abdomen 162 or elsewhere in the drone 160. A plurality of wings 174 are secured to the abdomen 162 corresponding to the aforementioned rotors 170. The wings 174 may be constructed of any of a variety of known or available films in a conventional manner so as to facilitate flight of the drone 160.

The drone 160 includes a head 176. A detailed depiction of the head 176 is presented with respect to FIG. 19. The head 176 includes video sensors 178 that are used in the manner described above with other embodiments to provide video and/or image data. As discussed above, this data is usable to assist in the navigation or navigation assistance of the drone 160 and/or to identify targets and/or hosts. UVC LEDs 180 are provided on either side of the head 176. While FIG. 19 depicts two LEDs on either side of the head 176, it will be recognized that in an alternative embodiment, the LEDs 180 are provided in a “compound eye” effect to increase the surface area and the directionality of the emitted UVC light energy from the drone 160.

The head 176 further includes an array of photo spectroscopic sensors 182. In certain embodiments, the head 176 further includes one or more laser diodes 184, which are also present in certain embodiments of the other devices discussed above.

Referring back to FIG. 18, the drone 160 is provided with an articulable tail 186. Actuators (not depicted) within the tail 186 enable movement of the tail 186, including a tail end 188 that is shown in greater detail in FIG. 20. The tail end 188 includes an array 190 of UVC LEDs 192. The tail end 188 further includes an LED indicator light 194. In operation, the articulable tail 186 can be moved to position the LEDs 192 to emit UVC wavelength light energy on the target to be disinfected. The LED indicator light 194 helps to visually identify the location of the end of the tail 186.

In exemplary embodiments, the drone 160 can be operated manually by remote, or autonomously by integrated computer hardware, software, and other communication and control systems including GPS and wireless communications, which is discussed further below. The drone 160 may be equipped with Digital Video Sensors, GPS Guidance Receiver, Wi-Fi receiver, Bluetooth, radio receiver, as well as laser photo diodes, fluoroscopic sensors, digital photo spectroscopic sensors, holographic sensors, etc. that allow the drones to complete a precision UVC scan and extermination of the surface of a 3 dimensional space. For example, these spaces may include a hospital room, a surgery room, a greenhouse, or a field. This allows the drones 160 to evenly track through the space according to the laser GPS guidance and/or preset commands to ensure proper exposure time of UVC light over surfaces to kill microorganisms in relation to the time and distance from the surface. This approach can ensure that every inch of exposed host surface, and every target (such as a microorganism) has been exposed to the precise intensity of UVC light, decreasing harmful microorganisms significantly.

In certain embodiments, the drone 160 incorporates GPS, cameras, and others sensors, to effectively “hunt” harmful targets (such as microorganisms) by a process of identification and extermination. The drone 160 constantly retrieves information being transmitted by the photo spectroscopic sensors, laser photo diodes, etc. about its environment, targets, and hosts. This streaming data may be stored in a database and/or analyzed in real time. Once a sample of a microorganism is identified as a potential target, the location can be identified within an extermination map and exterminated with UVC light. The drones 160 identify a microorganism through visual and/or chemical analysis and hunt it down, report its location, destroy it, scan the area and confirm the kill.

In further embodiments, UVC drones 160 can be operated individually or together with other drones 160 in swarm formations to ensure proper exposure of UVC light onto small and large surfaces or areas. Similarly, ground devices (such as drone 132) and aerial devices (such as drone 100) can work together in pairs, teams, swarms, etc. In certain embodiments, these devices can land on or dock with and be deployed or recharged with larger UVC drones or mobile transport vessel in remote applications. Land based drones can “pair up” with an aerial drone so each land based drone can recharge/transport aerial drone to and from deployment zones. In other words, an aerial drone may be configured to piggy back with a land based wheeled or tracked drone.

In still further exemplary embodiments, any of the aforementioned or other anticipated devices can have an antimicrobial surface on the exterior, such as nano silver or a material that is UVC photo catalytic, such as titanium dioxide. Incorporating such an exterior or exterior treatment increases the effects of the UVC light emissions and reduces the chance of the drone or device picking up or otherwise transporting/transmitting targets to other hosts, such as harmful microorganisms to other surfaces, areas, etc. In certain embodiments, any of the aforementioned devices may also or alternatively incorporate solar cells/films/or fabrics embedded into the architecture to recharge the batteries or otherwise obtain power through remote electromagnetic charging methods.

In further devices and methods for crop protection and other applications, land based and other drones as previously described have armatures (not depicted) that include UVC LED arrays. These armatures can be movably positioned to emit UVC light to reach larger areas, for example in hospitals, greenhouses and fields between the rows, and the like. In a greenhouse or field, the drone may be configured to track down a center row while emitting UVC light onto the rows of plants on either side of it, using these side arrays. The size, height, and intensity of such UVC LED arrays can be configured according to the species (height and size) growth phase, and level of infection or disease of the plant. Seedlings and small plants in fields, greenhouses, gardens, often require small drones with smaller UVC LED arrays and smaller armatures. Larger species plants, and more mature plants, often require larger drones with larger arrays and armatures. Moreover, these armatures allow for the LED arrays to be pointed down and in, onto the ground and base of the plants, surfaces, and other hosts, as well as up and out as the case may be. The armatures and arrays can also be pointed and tilted to ensure sufficient UVC light exposure to surfaces in all directions collectively, in a specific combination, or selectively to maximize effective exposure and/or minimize unintended exposure.

In a further embodiment of systems and methods for crop protection and other applications, decentralized systems of land and aerial drones work together to cover more area, more precisely and more efficiently than one centralized system. However, such situations may in certain embodiments require a more centralized, larger tracked or wheeled drone(s) that tracks through a space for crop protection/sterilization instead of smaller numerous drones. In certain embodiments, this larger drone also has a larger capacity for UVC exposure, either as an area or intensity. For example, a densely grown corn field in August would often require a larger tracked drone with large armatures and powerful arrays to cover the whole plant and to penetrate the leaf canopy. Once again, this is calculated and accommodated for in proportion to the species and number of targets (such as bugs and mold, for example), growth phase, and the like. If a field, classroom, greenhouse, or other area or host requires additional UVC light exposure to address a heightened level of harmful microorganisms, larger and/or more powerful drones and/or teams or swarms can be employed to overwhelm and eliminate the harmful microorganism(s), and also to scan the area in great detail to ensure the threat has not spread and is not present in an area previously undetected.

While the exemplary embodiments of disinfection or extermination devices provided herein have been described with the suggested use of crop and/or plant disinfection, it will be recognized that a variety of other uses are also anticipated. These other uses may include, but are not limited to, disinfection of rooms and/or surfaces, animal disinfection, as well as other antimicrobial purposes as will be recognized by a person of ordinary skill in the art.

In a further example, drones can use GPS, video, video sensor, Wi-Fi, and the like for guidance or mapping through use of an extermination map, which is discussed further below. To aid this process, and to ensure the precise mapping of the space (a field, greenhouse, warehouse, or hospital room, for example), sensors can be placed on the perimeter and/or throughout the space to give the drones more data, or a better picture of their environment. This peripheral and/or integrated sensor array can also be spectroscopic, fluoroscopic, and other sensors to provide the drones with a better picture of their environment biochemically, and to help them locate the harmful microorganisms or other targets. In certain embodiments, this process can be linked to a drone(s) as an “on call” sentry response deployed when one or more sensors identify and/or confirm the existence and location of the harmful microorganism.

In exemplary embodiments, such devices are used to disinfect surfaces to kill germs including but not limited to flu, strep, staph, or MRSA treatment resistant microorganisms.

In a still further exemplary embodiment, particularly within a health care or biohazard environment, UVC drones are deployed remotely and may be requested to disinfect a specific area. Exemplary embodiments of the drone as described herein may be programmed so as to perform an automatic scan of other known highly-contaminated or trafficked areas.

In exemplary embodiments the drones as described herein may be outfitted with cameras, sensors, diodes, laser, digital mass spectroscopy, or fluoroscopy capabilities to identify harmful organisms, emit the appropriate dosage of UVC light into the area, and confirm the kill through subsequent scanning.

In exemplary embodiments, the information processed by the cameras, sensors, and any other source can be sent to a central database or a platform where the information can be analyzed, cross referenced, validated, and stored. And this information can be relayed or accessed to review performance and efficacy data.

In an exemplary embodiment, any of the aforementioned devices are held a few inches off of the plant tissue, dirt, or other host to be disinfected with the device moved over and about the surface to eradiate the harmful microorganisms, diseased tissue, or other targets of interest on the host or hosts with UVC light. The UVC light can be focused or diffused using lenses and/or reflectors to change the angle and intensity of the UVC light. While an exemplary embodiment described herein as using batteries, those batteries may exemplarily be, but not limited to CR125 watch batteries, AAA, AA, C, D, 9 volt, or other recognized types and sizes of batteries. In still further exemplary embodiments, the disinfection device may be rechargeable via electricity or alternative energy. Such alternative energies may include a kinetic piston or solar charging solutions.

As described herein, certain exemplary embodiments include an indicator light that illuminates in a first color to indicate that the device is in an on or powered state, a second color to indicate that the device is emitting UVC wavelength light energy, a third color to indicate that the necessary UVC exposure has been delivered, and a fourth color to indicate the presence of some issue, for example, such as low battery or poor signal strength for communicating with remote controllers or other devices. A still further embodiment particularly resembles a hand-held flash light in which an array of more than one UVC LED is provided. In this example, one or more visible light spectrum emitting diodes may also be included such that the area eradiated with UV wavelength C energy is also illuminated with visible light such that the operator can discern the area receiving UVC light.

In exemplary embodiments, the device further includes settings and/or markings on the side of the device for adjusting the focus of the lens according to what type of UVC treatment the user is trying to administer. Broad and/or diffused focus of the lens may be used for maintenance services, while focused adjustment of the lens may be used for disinfection, such as of a crop. For example, daily scans of surfaces or objects may be paired with a diffused application, whereas focused intensity is needed, and/or for longer durations, to remediate densely concentrated areas of infection or contamination.

In still further exemplary embodiments, UVC wavelength light emitting diodes are incorporated to cover large areas of plants (or other potential hosts) using armigers, carts, farm implements, drones, or other devices to emit the UVC light onto the plant tissue to reduce harmful microorganisms that negatively affect the health of the plant tissue.

In exemplary embodiments of self-propelled drones, the drone includes a laser GPS guidance system to guide and operate flying or driving drones in the same way that newer tractors and combines are operated. These drones may further be equipped with sensors and diodes to monitor the health of the plant tissue before and after completing an extermination program on the target. These sensors and diodes may further identify and count the number of targets on a given host, for example. Moreover, the data regarding the health of the plant tissue, microorganism count, and other data processed by the camera and/or sensors, diodes, lasers, digital mass spectroscopy, or fluoroscopy can be sent to a central database or platform where information is analyzed, cross referenced, monitored and trended, displayed to the user, validated, and stored. This information can also be relayed or accessed to review performance and efficacy data to develop extermination schedules, new extermination programs, new device and/or extermination capabilities and the like.

As will be discussed further below, the precise exposure of UVC light is preferably determined and delivered to at least a minimum level sufficient to ensure extermination of a target to a predetermined level of confidence, such as based on statistical models incorporating the target type, intensity, and exposure time of UVC light. The operational examples provided herein may be applicable to the operation of any of the UVC devices as described above, for example with respect to FIGS. 1-20. Additionally, a person of ordinary skill in the art will recognize further devices from the present disclosure that may be operated in the manners as described herein. In exemplary embodiments, a UVC delivery device is operated to not surpass a maximum exposure as a predetermined excessive level of UVC light that would harm the host, while providing a sufficient dose so as to kill the targeted pest. This may exemplarily be provided and/or determined from statistical models for effective levels of UVC exposure. However, in practical use some amount of UVC overexposure harm to the host may occur, particularly if the minimum and maximum doses as determined in the present method are close, or if a variety of hosts and target pests are to be treated simultaneously. However, such maximum values for UVC light exposure may nonetheless prevent harm beyond that which is required to exterminate the target.

This exemplifies one significant advantage of the devices and methods presently disclosed, whereas devices and methods known in the art cannot ensure that the target receives a sufficient level of UVC light to ensure extermination, and/or that exposure is limited to not excessively damage the host. The interaction between UVC light and the host can be complex with different dosages of UVC having positive or deleterious effects. Additional description of some of these effects is provided by Bridgen, M. P. (2016). Using ultraviolet-C (UV-C) irradiation on greenhouse ornamental plants for growth regulation. Acta Hortic. 1134, 49-56 DOI: 10.17660/ActaHortic.2016.1134.7, which is herby incorporated by reference in its entirety. Certain devices and methods known in the art simply suggest a certain exposure time for delivering UVC light that is assumed to be well beyond what is required to exterminate the target. However, these methods fail to account for the particular target type and intensity of UVC light, and do not provide any mechanism for preventing excess exposure to the host.

In addition to excess UVC light exposure harming the host, this practice provides further disadvantages, particularly for widespread application. For example, providing excess exposure of UVC light to a particular target necessarily results in excessive time spent treating that particular target, and thus an extended time for treating an overall area or multiple targets. This alone may preclude use of UVC light as a means for extermination over large areas, such as a field of crops, whereby the excessive time spent wasted on each target may be multiplied by hundreds of thousands of hosts and potentially millions of particular targets corresponding thereto. Moreover, in devices and methods incorporating a battery operated UVC emitter or a battery powered self-propelled device carrying such a UVC emitter, this wasted generation of UVC light only reduces the capacity of the battery to cover a desired area, once again precluding the use of such devices in many industries and for many applications. In contrast, the presently disclosed devices and methods ensure the precise amount of UVC light is delivered to ensure the objective of extermination, without waste.

UV dose is exemplarily represented in millijoules per centimeter squared (mJ/cm̂2) or the equivalent joules per meter squared (J/m̂2). Dose is related to the intensity of the UV exposure and the length of time of the exposure. UV intensity is also known as UV irradiance and is exemplarily represented in milliwatts per centimeter squared (mW/cm̂2). When UV intensity is considered along with an exposure time in seconds, the same UV dose can be achieved for example with higher intensity at a shorter exposure time or a lower intensity with a longer exposure time.

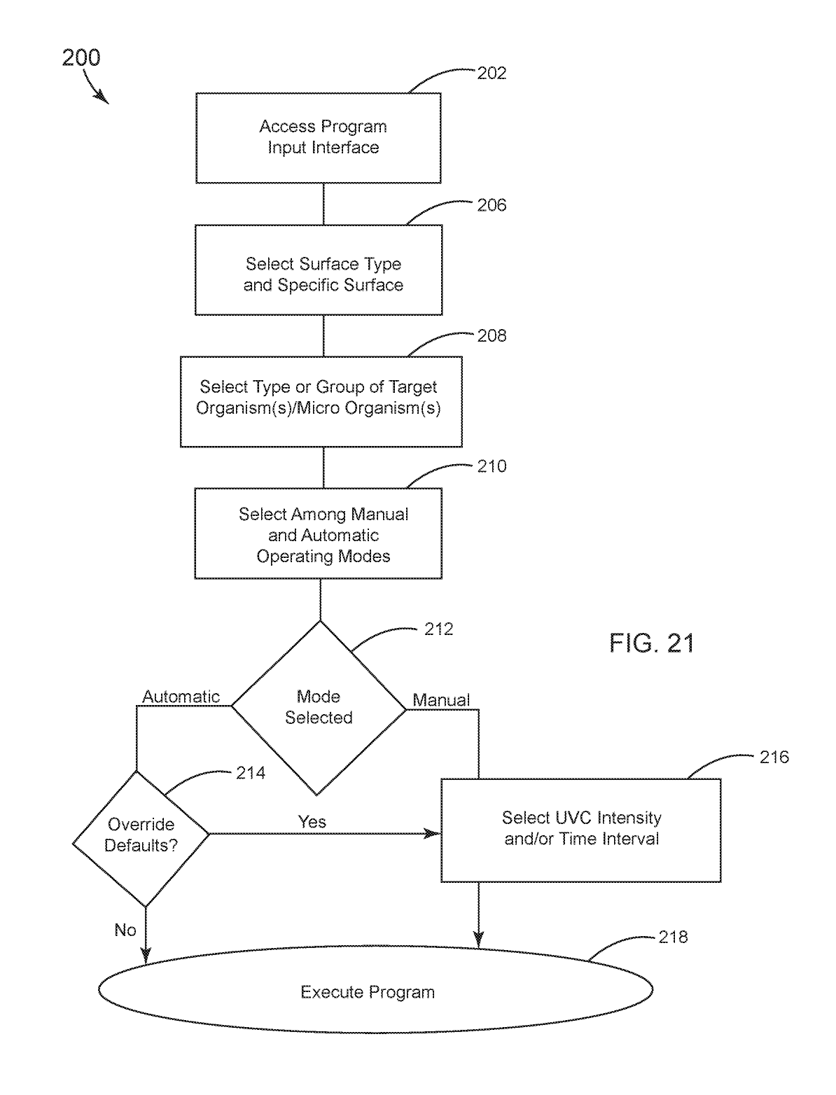

Various methods for exterminating targets have been described herein, including through use of a wide variety of devices for emitting UVC light. FIG. 21 depicts an exemplary method 200 in flowchart form for executing such an extermination program. In the embodiment shown, the user accesses a user input interface in step 202, for example through the input interface 412 shown in FIG. 23. In certain embodiments, the input interface 412 is a touchscreen, though other embodiments, such as a series of selector switches, dials, sliders, or knobs, are also anticipated by the present disclosure. Through the input interface 412, the user selects one or more host types, and optionally specific hosts within those host types, in step 206. These host types and specific hosts define the hosts that the user wishes to disinfect, or to exterminate the target from. For example, the host type may be some form of tissue, such as that belonging to an animal or plant. Alternatively, the host type may be an inanimate object, such as a countertop or operating table. The host type selected in 206 is used to define a maximum UVC dose. It will be recognized that depending upon the host, that host will have a resistance to UVC exposure. In exemplary embodiments, an inanimate object (e.g. an operating table, a counter, a wall or a floor) may have a high resistance to UVC dose, and the resulting maximum UVC bound based upon the selected host type may be similarly high, for example the maximum output of the UVC delivery device. In other embodiments, a UV sensitive material, e.g. fabrics or some plastics, animals, or plants may be susceptible to damage from UVC dose, in embodiments where such a host is selected, the maximum UVC bound will be set lower in accordance with the resistance of the host. The resistance of the host may be provided in a look-up table or other data storage accessible by the system upon the selection of the host type.

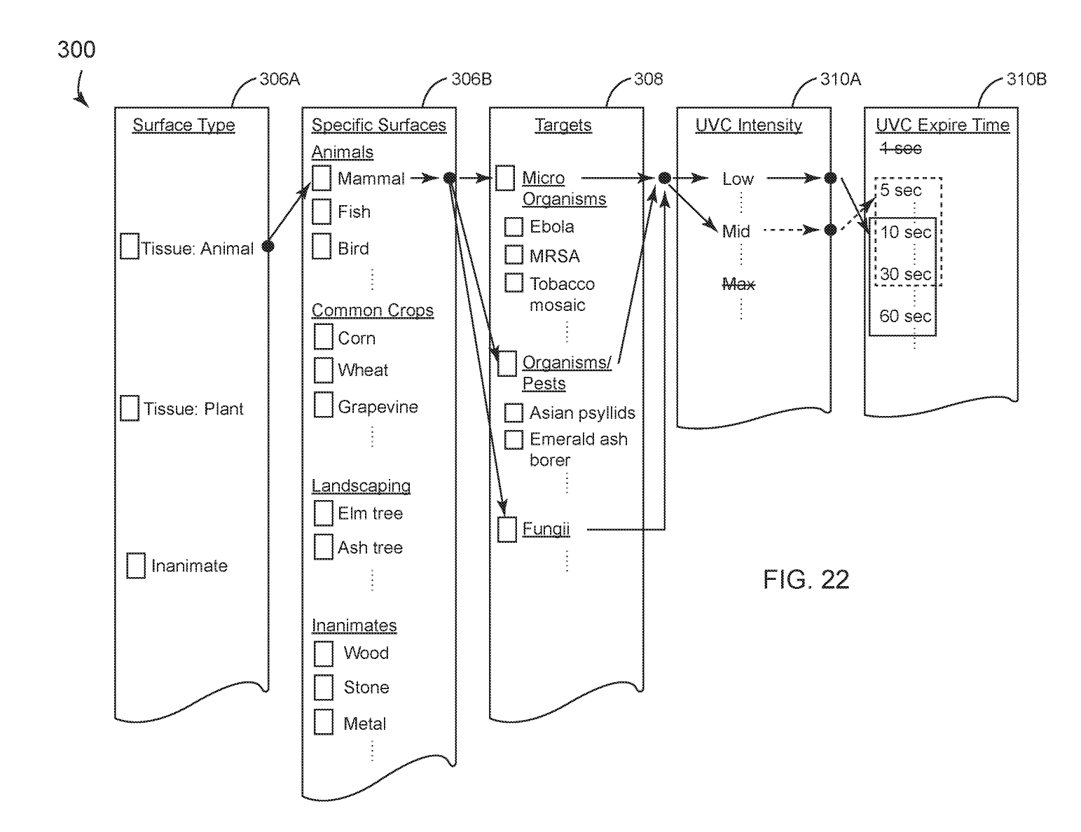

FIG. 22 depicts an exemplary data structure within a program 300. The selection of the host type is made through accessing the program 300 data within the host type category 306A. Following the selection of one or more host types in the host type category 306A, the user may optionally identify specific hosts listed in the specific hosts category 306B. In certain embodiments, the data structure provided in the program 300 is “smart,” whereby the particular selection of the host type category 306A results in a restricted listing of selections available for the specific hosts category 306B. For example, selection of the host type “tissue: animal” results in a listing of the available sub-options of animals (or specific tissues thereof) under the specific hosts category 306B. Within the specific hosts category 306B, the user may select a broad category, such as mammals, or may provide greater specificity, such as selecting an elephant versus a mouse as the host. Following the selection of host type and specific host in step 206, the user selects a type or group of targets in step 208 to be exterminated from these hosts. It should be recognized that while the selection of a host type is in certain embodiments incorporated into the determination of an intensity and exposure time for UVC light, the selection does not necessarily preclude the device from exterminating a target from a non-selected host type. However, in advanced operating modes incorporating sensors for detecting hosts and targets, certain extermination programs intentionally limit extermination to only those targets that are expressly selected, and/or only those that are present on expressly identified hosts (or, alternatively, in accordance with a list of restricted targets and/or hosts to not treat). In the inverse of the host type selection, the target type selection defines the minimum UVC dose provided by operation of the UVC delivery device. This is the minimum necessary effective dose and, as will be described in further detail herein, the effective dose is related not only to the target pest itself, but also to a desired log inactivation of the target pest. The operation of the UVC delivery device may still include a variety of operational settings to achieve the identified minimum necessary effective dose, for example due the intensity of the UVC source, and the exposure time from the UVC source.

As shown in FIG. 22, the targets category 308 may once again be smartly populated based on the selection of host type category 306A and/or specific hosts category 306B to limit the number of options listed to the user. For example, the selection of “mammals” within the specific hosts category 306B may populate the targets category 308 to include the categories of microorganisms, organisms/pests, and fungi, for example. Once again, the user may identify one or more particular targets, or one or more groupings of targets, such as microorganisms. Moreover, in the example of selecting “mammals,” certain embodiments would exclude targets that do not correspond to mammals.

In the method 200 shown in FIG. 21, the user further selects among options of manual and automatic operating modes in step 210. Among other parameters, the selection of manual or automatic operating modes includes the selection of UVC intensity 310A and UVC exposure 310B, as shown in FIG. 22. In manual mode, these particular selections are made by the user, though some of the options capable of being emitted by the device may be excluded based on the host type category 306A, specific hosts category 306B, and/or targets category 308. For example, FIG. 22 depicts a configuration in which the selection of a “mammal” as the specific host category 306B, regardless of the selected targets category 308, precludes the selection of a UVC intensity 310A classified as “max,” which in this embodiment is determined to be hazardous to the host, the mammal. For example, “max” may be the maximum possible output of the UVC light emitter, (for example, 25 watts).

Likewise, the UVC exposure time 310B is shown to be varied based on the selection of UVC intensity 310A, whereby a low intensity (for example, 1 watt) corresponds to a longer exposure time to achieve a particular UV dose, and vice versa. However, in each case, the lowest UVC exposure time 310B of one second is not selectable in the present example, either because the UVC intensity 310A of “max” is unavailable, and/or because the targets category 308 or the specific hosts category 306B require more exposure to ensure effective extermination of the targets. Limitations may also be placed based on the operational limits of the UVC light emitter, including actual or projected temperature. In certain embodiments, the selections of UVC intensity 310A and/or UVC exposure time 310B are not literally unselectable by the user, but are instead indicated as being non-recommended selections. In the event that a non-recommended selection is made, such as choosing “max” for the UVC intensity 310A in the example shown in FIG. 22, the UVC exposure time 310B may appropriately adjust in certain embodiments to correspondingly show shorter exposure times in recognition of the increased intensity.

In certain embodiments, the selection of an automatic operating mode in step 210 result in the automatic selection of UVC intensity 310A and/or the UVC exposure time 310B, rather than requiring the user to make such selection. In certain embodiments, the device incorporates saved preferences to inform the selections made in the automatic operating modes, such as a preference for speed. In such a case, the automatic operating mode will result in a selection of UVC intensity 310A to provide the minimum UVC exposure time 310B that nonetheless provides effective extermination of the selected target.

As previously stated, the recommended or automatic selections of UVC intensity 310A and UVC exposure time 310B may be based upon statistical modeling for effective extermination of targets, including any impact of being on the select host. In this regard, the intensity and exposure time of UVC light is based at least in part on the host type selected and the target type selected, which together define the extermination program.

In certain embodiments, including those incorporating statistical modeling, a minimum value of exposure is determined such that the target is exterminated to a predetermined level of confidence, which may vary depending on the application. For example, in a hospital setting, the minimum value may be set such that target elimination is expected with a confidence level of 99.99%. In contrast, a less-critical context such as a large field of wheat hosting a target that only mildly impacts yield may have a minimum value corresponding to extermination to a level of confidence of 90%, for example. The difference between 90% and 99.99% may be double or more the exposure time or intensity, depending on the particular target and/or host. Therefore, in a further embodiment, a user input of a desired target reduction may be input while in other embodiments if a specific target reduction is not input, then a default target reduction level is used. In exemplary embodiments, the target reduction is represented as a log-scale reduction of the target. It will be noted from the above, that each target and/or host has its own response to UVC exposure and therefore, will experience a different log inactivation of the target to a UVC dose. Therefore, the UVC dose selected in the methods as described herein are so selected to achieve a target log inactivation of the target pest.

Likewise, the maximum value of exposure may be determined such that the host is not exposed to a predetermined excessive level of UVC light that would harm the host, or would not harm the host more than required to properly exterminate the target. In this regard, a host that is susceptible to damage from UVC light may be prevented from overexposure, once again based on statistical modeling that such harm would occur. If the selected host type is chosen from the host type category 306A as being inanimate, there may be no maximum value provided if the host is not susceptible to harm. However, as previously described, the automatic operating mode may nonetheless limit the emission of UVC light for other reasons, such as to provide for expeditious extermination, and/or to preserve battery life for the device. Exemplary embodiments of various UV dosages for varying log inactivation of target pests are provided in tables found in “Ultraviolet Light Disinfection Data Sheet”, ClorDiSys Solutions Inc. 2014 and “Application Note: UVC LEDs for Disinfection” Crystal IS, Inc., 2014, both of which are incorporated herein by reference in their entireties.

In certain embodiments, even the selection of an automatic operating mode in step 210 permits the user to nonetheless override its defaults in step 214, allowing the user to select the UVC intensity and/or exposure time in step 216. These override selections may include the same or a limited subset of options relative to operation in the manual operating mode. Once the parameters have been selected, the device is configured to execute the extermination program in step 218, whether as a hand-held device, drone, or other device configured to emit UVC light in accordance with the present disclosure. The extermination program thus includes the time and intensity of operation of the UVC delivery device that achieves a UVC dose for the desired log inactivation of the target pest within the dose bounds of the minimum to achieve the desired log inactivation of the target pest and the maximum to which the host can be exposed without significant harm, or an otherwise safe exposure to the host.

Exposure time relative to achieve the UVC dose as described above can be controlled in the extermination program in a number of ways, some of which may depend upon the UVC delivery device used, for example any of the UVC delivery devices described in the present application. In a hand held device, for example as depicted and described with respect to FIGS. 1-12, an assumption may be made that the user will point the device at the object or surface to be treated, and the extermination program will operate the device at the determined intensity for the time duration to achieve the determined UVC dose, before turning off or providing the user with a notice (e.g. audio or visual) to direct the UVC light from the device to another area/position for treatment. In embodiments that include one or more drones 100 as exemplarily depicted and described with respect to FIGS. 13-20, the drone may move itself or an array of UVC LEDs or an armature with an array of UVC LEDs when a target area of the host has been exposed with the determined time at the determined UVC LED intensity to achieve the UVC dose.

FIG. 23 depicts one exemplary control system 400 for operating a device to exterminate a target on a host using ultraviolet light as described herein. In the embodiment shown, the primary controller 450 includes an input/output module 454, a processing module 456, and a memory module 458 configured to communicate therebetween to execute the extermination program previously discussed, which is stored in the memory module 458 as program 460. The primary controller 450 also communicates with various inputs 410 and outputs 480. It should be recognized that the inputs 410 may also constitute outputs, and the outputs 480 may also constitute inputs relative to communications to and from the primary controller 450.

It should be recognized that any such functional and/or block components and processing steps discussed and depicted herein may be realized by any number of hardware, software, and/or firmware components configured to perform the specified functions. For example, certain embodiments employ various integrated circuit components, such as memory elements, digital signal processing elements, logic elements, look-up tables, or the like, which are configured to carry out a variety of functions under the control of one or more processors or other control devices. The connections between functional and logical block components are also merely exemplary. Moreover, the present disclosure anticipates communication among and between such components being wired, wireless, and through different pathways.

These functions may also include the use of computer programs that include processor-executable instructions, which may be stored on a non-transitory tangible computer readable medium. The computer programs may also include stored data. Non-limiting examples of the non-transitory tangible computer readable medium are nonvolatile memory, magnetic storage, and optical storage. As used herein, the term module may refer to, be part of, or include an application-specific integrated circuit (ASIC), an electronic circuit, a combinational logic circuit, a field programmable gate array (FPGA), a processor (shared, dedicated, or group) that executes code, or other suitable components that provide the described functionality, or a combination of some or all of the above, such as in a system-on-chip. The term module may include memory (shared, dedicated, or group) that stores code executed by the processor. The term code, as used herein, may include software, firmware, and/or microcode, and may refer to programs, routines, functions, classes, and/or objects. The term shared, as used above, means that some or all code from multiple modules may be executed using a single (shared) processor. In addition, some or all code to be executed by multiple different processors may be stored by a single (shared) memory. The term group, as used above, means that some or all code comprising part of a single module may be executed using a group of processors. Likewise, some or all code comprising a single module may be stored using a group of memories.

In the exemplary embodiment shown, the inputs 410 include the input interface 412 previously discussed which permits the user to select the host type, target type, and in certain embodiments, the intensity and exposure time of UVC light to exterminate the target. Other options and selections may also be made through the input interface 412, as previously discussed. The inputs 410 further include target detection sensors 414, distance sensors 416, environmental sensors 418, and exposure sensors 420 as previously discussed. Target detection sensors 414 may include fluoroscopic sensors, distance sensors 416 may include laser photodiodes, and environmental sensors 418 exemplarily include GPS systems, for example, each having been discussed in greater detail above. Exposure sensors 420 may also be incorporated, which beyond detecting the presence of a target after completing an extermination program may be configured to detect the real-time emission of UVC light. For example, a photodiode may be incorporated as an exposure sensor 420 as an accuracy check to determine that the UVC light being emitted matches the UVC light intended to be emitted.

The primary controller 450 further communicates with a plurality of outputs 480. These outputs 480 exemplarily include a UVC controller 482, which among other things controls the power state, voltage, and amperage delivered to the UVC emitter to generate UVC light. The UVC controller 482 may also perform other aspects of operation, for example to vary the intensity of a UVC light emitter over the exposure time, such as necessary to optimize the extermination of a target, or to extend the life of the UVC light emitter in service. Likewise, the vehicle controller 484 may receive commands from the primary controller 450, particularly with respect to self-propelled devices configured for delivering the UVC light. Various controls for operating the drones and other devices have been previously discussed and would be known to one of ordinary skill in the art, including various controls for flying or driving a device, and for ensuring operational safety in use.

The outputs 480 of the present embodiment further include an external monitoring module 486 that receives data collected and processed within the primary controller 450, as previously discussed. This may provide trending of delivered UVC light for a particular target or area over time or be used for scheduling and optimization of future UVC light treatments. Similarly, the primary controller 450 communicates with a display device 488, which may be on the device itself or remote, to provide the user with real-time and/or historic information relating to extermination and use of the UVC device. In certain embodiments, the display device 488 is combined with the input interface 412, such as in the case of a touchscreen. However, particularly with smaller devices, the display device 488 may be a remote unit that is used to make the necessary selections and programming previously discussed, which are later communicated with the UVC light emitting device having a separate input interface 412 thereon.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives and modifications are possible within the scope of the appended claims.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

I claim:

1. A method for exterminating a target on a host using ultraviolet (UVC) light, the method comprising:
providing a device configured for emitting UVC light for exterminating the target;
selecting a host type from a plurality of host types for the target to be exterminated from;
selecting a target type from a plurality of target types for the target to be exterminated;
assigning an intensity and an exposure time of UVC light to exterminate the target, wherein the intensity and the exposure time are based at least in part on the host type selected and the target type selected, and wherein the intensity and exposure time together define an extermination program; and
controlling the device to emit UVC light to exterminate the target according to the extermination program.

2. The method according to claim 1, further comprising indicating when the device has completed the extermination program.

3. The method according to claim 1, further comprising automatically assigning the intensity and exposure time based at least in part on the host type selected and the target type selected.

4. The method according to claim 1, further comprising determining at least one of a minimum value and a maximum value for assigning at least one of the intensity and the exposure time based on the host type selected and the target type selected.

5. The method according to claim 4, wherein the minimum value is determined such that the target is exterminated to a predetermined level of confidence.

6. The method according to claim 4, wherein the maximum value is determined such that the host is not exposed to a predetermined excessive level of UVC light.

7. The method according to claim 1, further comprising automatically detecting and exterminating the target when the target matches the selected target type.

8. The method according to claim 1, wherein the device is self-propelled, further comprising assigning an extermination map defining where the device will exterminate the target.

9. The method according to claim 1, wherein the host is a plurality of hosts, and wherein the target is a plurality of targets on the plurality of hosts, further comprising detecting with a sensor the plurality of targets on the plurality of hosts to be exterminated based on the target type and the host type selected, and further comprising controlling the device to automatically emit UVC light at a given target among the plurality of targets until the sensor no longer detects the given target.

10. The method according to claim 1, wherein the device is handheld and includes a user input for selecting the target type, further comprising recommending the intensity and the exposure time for the extermination program, and further comprising customizing the extermination program as a custom extermination program and saving the custom extermination program for future use.

11. The method according to claim 1, wherein the device is a drone configured for emitting UVC light between 240 and 280 nanometers in wavelength using one or more LEDs, wherein the host is an agricultural crop, and wherein the target is powdery mildew.

12. A device for exterminating a target on a host using ultraviolet (UVC) light, the device comprising:
a UVC emitter configured for emitting UVC light for exterminating the target;
a memory module that stores a plurality of host types for the target to be exterminated from, a plurality of target types for the target to be exterminated, and a plurality of intensities and exposure times for the UVC emitter to emit UVC light;
a user input module for selecting a host type from the plurality of host types and a target type from the plurality of target types;
a processing module that assigns an intensity and an exposure time of UVC light from the plurality of intensities and exposure times, respectively, based at least in part on the host type selected and the target type selected, wherein the intensity and the exposure time of UVC light assigned together define an extermination program for exterminating the target;
a controller module that controls the device to emit UVC light to exterminate the target according to the extermination program; and
an indicator that indicates when the device has completed the extermination program.

13. The device according to claim 12, wherein the processor module is configured to automatically recommend a range of intensities and a range of exposure times for assigning the intensity and exposure time of UVC light, and wherein the user input module is configured to select the intensity and the exposure time of UVC light from the range of intensities and the range of exposure times to be assigned by the processing module.

14. The device according to claim 12, wherein the processing module is configured to prevent assigning an undesirable intensity from the plurality of intensities and an undesirable exposure time from the plurality of exposure times based at least in part on the host type selected and the target type selected.

15. The device according to claim 14, wherein the undesirable intensity and the undesirable exposure time are determined by the processor to correspond to at least one of the target not being exterminated to a predetermined level of confidence and the host being exposed to a predetermined excessive level of UVC light.

16. The device according to claim 12, further comprising a plurality of sensors that detect the target, wherein the controller module controls the device to exterminate the target detected when the target matches the selected target type.

17. The device according to claim 12, wherein the device is self-propelled, further comprising an extermination map module that defining where the device will exterminate the target.

18. The device according to claim 12, wherein the host is a plurality of hosts, and wherein the target is a plurality of targets on the plurality of hosts, further comprising detecting with the plurality of sensors the plurality of targets on the plurality of hosts to be exterminated based on the target type and the host type selected, and further comprising the controller module being configured to control the device to automatically emit UVC light at a given target among the plurality of targets until the plurality of sensors no longer detect the given target.

19. The device according to claim 12, wherein the device is handheld and includes a user input for selecting the target type, wherein the UVC emitter is one or more LEDs, wherein the extermination program is customizable as a custom extermination program, and wherein the memory module is further configured to save the custom extermination program for future use.

20. The device according to claim 12, wherein the device is a drone configured for emitting UVC light between 240 and 280 nanometers in wavelength, wherein the host is an animal, and wherein the target is white flies.
